(19) United States
(12) Patent Application Publication
(10) Pub. No.: US 2022/0096387 A1
(43) Pub. Date: Mar. 31, 2022

(54) SUBLINGUAL DELIVERY FOR MITIGATION OF SIDE EFFECTS ASSOCIATED WITH LURASIDONE

(71) Applicant: Vivera Pharmaceuticals Inc., Newport Beach, CA (US)

(72) Inventors: Paul Edalat, Newport Beach, CA (US); Stephen McColgan, Newport Beach, CA (US); Gerald Anthony Maguire, San Juan Capistrano, CA (US)

(73) Assignee: Vivera Pharmaceuticals Inc., Newport Beach, CA (US)

(21) Appl. No.: 17/545,391

(22) Filed: Dec. 8, 2021

Related U.S. Application Data
(63) Continuation-in-part of application No. 17/495,666, filed on Oct. 6, 2021.
Application No. 17/495,666 is a continuation-in-part of application No. 17/464,587, filed on Sep. 1, 2021.
Application No. 17/464,587 is a continuation-in-part of application No. 15/613,057, filed on Jun. 2, 2017.
Application No. 15/613,057 is a continuation of application No. 14/760,311, filed on Jul. 10, 2015, now abandoned.
(60) Provisional application No. 61/937,021, filed on Feb. 7, 2014.

(51) Int. Cl. A61K 9/48; A61K 9/00; A61K 45/06

(52) U.S. Cl. CPC A61K 9/4808; A61K 45/06; A61K 9/4825; A61K 9/006

(57) ABSTRACT

Sublingual delivery vehicles (SDVs) including tablets and gel strips may mitigate or eliminate side effects associated with active ingredients included in the SDVs. An exemplary SDV may include: an ingredient mixture including a flavoring agent and a lubricant; and a specified dose of lurasidone, wherein the SDV dissolves within thirty seconds of sublingual administration. A method of manufacturing an SDV may include mixing a set of ingredients, wherein the set of ingredients includes: a flavoring agent, a lubricant, and a specified dose of lurasidone; and forming the SDV from the mixed set of ingredients. A method for treating mental disorders may include administering, once a day, an ingredient mixture including: a flavoring agent; a lubricant; and a specified dose of lurasidone, wherein the ingredient mixture is administered via a sublingual delivery vehicle (SDV).

### CROSS-REFERENCE TO RELATED APPLICATIONS

This application is a continuation-in-part of U.S. patent application Ser. No. 17/495,666, filed on Oct. 6, 2021. U.S. patent application Ser. No. 17/495,666 is a continuation-in-part of U.S. patent application Ser. No. 17/464,587, filed on Sep. 1, 2021. U.S. patent application Ser. No. 17/464,587 is a continuation-in-part of U.S. patent application Ser. No. 15/613,057, filed on Jun. 2, 2017. U.S. patent application Ser. No. 15/613,057 is a continuation of U.S. patent application Ser. No. 14/760,311, filed on Jul. 10, 2015. U.S. patent application Ser. No. 14/760,311 is a national stage entry of PCT Patent Application serial number PCT/US2014/022054, filed on Mar. 7, 2014. PCT Patent Application serial number PCT/US2014/022054 claims priority to U.S. Provisional Patent Application Ser. No. 61/937,021, filed on Feb. 7, 2014.

### BACKGROUND

Pharmaceutical, supplement-based, and nutraceutical markets seek safer and more efficient ways to deliver active ingredients for treatment of conditions such as chronic mental health disorders. Such active ingredients may be associated with various undesirable side effects.

Therefore, there is a need for optimized ways to deliver active ingredients.

### BRIEF DESCRIPTION OF THE SEVERAL VIEWS OF THE DRAWING

The novel features of the disclosure are set forth in the appended claims. However, for purpose of explanation, several embodiments are illustrated in the following drawings.

FIG. 1A illustrates an example of one or more embodiments described herein, in which a sublingual tablet has a convex shape;

FIG. 1B illustrates an example of one or more embodiments described herein, in which a sublingual tablet has a round concave shape;

FIG. 1C illustrates an example of one or more embodiments described herein, in which a sublingual tablet has an oval concave shape;

FIG. 1D illustrates an example of one or more embodiments described herein, in which a sublingual tablet has a curved oval concave shape;

FIG. 2A illustrates an example of one or more embodiments described herein, in which a sublingual capsule includes a casing and extruded filling;

FIG. 2B illustrates an example of one or more embodiments described herein, in which a sublingual capsule includes a liquid or gel filling;

FIG. 2C illustrates an example of one or more embodiments described herein, in which a sublingual capsule includes a dry powder filling;

FIG. 3A illustrates an example of one or more embodiments described herein, in which an offset extruded sublingual strip includes a casing and extruded filling;

FIG. 3B illustrates an example of one or more embodiments described herein, in which an offset extruded sublingual strip includes a liquid or gel filling;

FIG. 3C illustrates an example of one or more embodiments described herein, in which an offset extruded sublingual strip includes a dry powder filling;

FIG. 4A illustrates an example of one or more embodiments described herein, in which a sublingual capsule extrusion is generated;

FIG. 4B illustrates an example of one or more embodiments described herein, in which a sublingual strip extrusion is generated;

FIG. 5A illustrates an example of one or more embodiments described herein, in which waffle gel strips include ingredient fillings;

FIG. 5B illustrates an example of one or more embodiments described herein, in which dimpled gel strips include ingredient fillings;

FIG. 6A illustrates an example of one or more embodiments described herein, in which a sheet of uncut waffle gel strips is ready to be filled;

FIG. 6B illustrates an example of one or more embodiments described herein, in which a sheet of uncut waffle gel strips includes fillings;

FIG. 6C illustrates an example of one or more embodiments described herein, in which a sheet of uncut waffle gel strips includes a gel cover;

FIG. 7A illustrates an example of one or more embodiments described herein, in which a sheet of waffle gel strips is imprinted and filled;

FIG. 7B illustrates an example of one or more embodiments described herein, in which a sheet of waffle gel cells has been cut into strips;

FIG. 8A illustrates an example of one or more embodiments described herein, in which a bubbled gel mixture strip rope is generated;

FIG. 8B illustrates an example of one or more embodiments described herein, in which a bubbled gel mixture strip rope is has been cut into strips;

FIG. 9 illustrates an example of one or more embodiments described herein, in which an ingredient listing is provided for a sublingual olanzapine and metformin product;

FIG. 10 illustrates an example of one or more embodiments described herein, in which an ingredient listing is provided for a sublingual trazodone product;

FIG. 11 illustrates an example of one or more embodiments described herein, in which an ingredient listing is provided for a sublingual sildenafil citrate product;

FIG. 12 illustrates an example of one or more embodiments described herein, in which an ingredient listing is provided for a sublingual blonanserin product;

FIG. 13 illustrates an example of one or more embodiments described herein, in which an ingredient listing is provided for a sublingual lurasidone product;

FIG. 14 illustrates an example of one or more embodiments described herein, in which an ingredient listing is provided for a sublingual vortioxetine product;

FIG. 15 illustrates an example of one or more embodiments described herein, in which an ingredient listing is provided for a sublingual brexpiprazole product;

FIG. 16 illustrates a flow chart of an exemplary process that produces sublingual tablets;

FIG. 17 illustrates a flow chart of an exemplary process that extrudes sublingual capsules;

FIG. 18 illustrates a flow chart of an exemplary process that produces sublingual gel strips; and

FIG. 19 illustrates an exemplary treatment schedule that uses sublingual products of some embodiments.

### DETAILED DESCRIPTION

The following detailed description describes currently contemplated modes of carrying out exemplary embodiments. The description is not to be taken in a limiting sense, but is made merely for the purpose of illustrating the general principles of some embodiments, as the scope of the disclosure is best defined by the appended claims.

Various features are described below that can each be used independently of one another or in combination with other features. Broadly, some embodiments generally provide ways to deliver various active ingredients, or “active pharmaceutical ingredients” (APIs), or “drugs”, via sublingual absorption. Delivery via sublingual absorption may reduce or eliminate side effects and may allow for lower dosages than required for other deliver methods (e.g., oral tablet or capsules). Various sublingual delivery vehicles (SDVs) may be used, such as tablets, capsules, gel strips, etc.

The active ingredients in some embodiments may include, for example, olanzapine. Olanzapine is an atypical antipsychotic medication that may be used for ongoing treatment of conditions such as bipolar disorder and schizophrenia. Olanzapine may be used to treat other conditions such as stuttering, major depressive disorder, anorexia, and other central nervous system (CNS) conditions, among others. Olanzapine may be associated with side effects including metabolic issues such as weight gain, hyperglycemia, and lipid elevations and may contribute to development of diabetes and/or exacerbate the symptoms of patients suffering from diabetes. Olanzapine may be used in dosages starting between two-and-a-half milligrams to twenty milligrams per day with a target of ten milligrams to fifteen milligrams per day when taken orally. Oral and orally disintegrating olanzapine tablets may be provided in dosages ranging from two-and-a-half milligrams to twenty milligrams. Olanzapine may typically be taken once daily (e.g., a single oral tablet may be ingested each day). Sublingual delivery of olanzapine, via the SDVs of some embodiments, may allow lower dosages to be utilized.

As another example, the active ingredients in some embodiments may include metformin. Metformin is a medication for the treatment of type II diabetes, particularly in people who are overweight. Side effects such as headaches, diarrhea, nausea, vomiting, flatulence, abdominal pain, and low blood sugar, among others, may be associated with use of metformin. Metformin may be used in dosages starting at five hundred milligrams twice per day, potentially increasing to one thousand milligrams twice per day when taken orally. Oral tablets may be provided in dosages ranging from five hundred to one thousand milligrams. Metformin may typically be taken twice daily (e.g., a single oral tablet may be ingested two times each day). Sublingual delivery of metformin, via the SDVs of some embodiments, may allow lower dosages to be utilized.

In some embodiments, the active ingredients may include a combination or mixture of olanzapine and metformin. Such a combined treatment may be associated with a daily treatment or dosing schedule.

Sublingual delivery of olanzapine and metformin is associated with reduction or elimination of side effects such as weight gain, hyperglycemia, nausea, abdominal pain, other gastrointestinal issues, and other side effects due to faster dissolving and absorption, among other factors. In addition, sublingual delivery may allow lower dosages to be used.

Because weight gain and diabetes are associated with use of olanzapine, the combination of olanzapine and metformin allows for a single combined treatment of the associated conditions. Metformin is associated with decreased insulin resistance and mitigation of metabolic side effects of olanzapine.

As another example, the active ingredients in some embodiments may include trazodone. Trazodone is an antidepressant medication that may be used to treat conditions such as major depressive disorder or anxiety disorders. Trazodone may be used to treat conditions such as insomnia. Trazodone may be associated with side effects ranging from dry mouth, vomiting, and headache to suicide, mania, and irregular heartbeat, among others. Trazodone may be used in dosages starting at fifty milligrams per day. The dose may be increased gradually (e.g., by fifty milligrams per day every three to four days) to a maximum dose up to four hundred milligrams per day for outpatients or six hundred milligrams per day to inpatients. Trazodone may be taken in divided doses throughout the day. After an adequate response is achieved, the dosage may be gradually reduced (or otherwise adjusted) depending on therapeutic response.

Trazodone tablets may be provided in dosages ranging from fifty milligrams to three hundred milligrams. Sublingual delivery of trazodone is associated with reduction or elimination of side effects such as dry mouth, vomiting, headache, and irregular heartbeat due to faster dissolving and absorption, among other factors. In addition, sublingual delivery may allow lower dosages to be used.

Furthermore, sublingual delivery allows quicker absorption that results in more rapid onset of sleep when used to treat insomnia. Faster elimination may also reduce daytime sleepiness associated with trazodone use. In addition, by bypassing the first-pass metabolism, sublingual products including trazodone may diminish adverse events by decreasing active metabolite meta-chlorophenylpiperazine (mCPP) which can be associated with conditions such as anxiety.

As still another example, the active ingredients in some embodiments may include blonanserin. Blonanserin is an atypical antipsychotic medication that may be used to treat conditions such as schizophrenia, bipolar disorder, stuttering, Tourette Syndrome, and major depressive disorder, among others. Blonanserin may be associated with side effects including weight gain. Blonanserin may affect cholesterol and triglyceride levels, and glucose and other blood lipid levels, among others. Blonanserin may be used in dosages starting at two to eight milligrams per day. Blonanserin may be taken in divided doses throughout the day (e.g., four milligrams twice per day).

Blonanserin tablets may be provided in dosages ranging from two milligrams to twenty milligrams. Sublingual delivery of blonanserin is associated with reduction or elimination of side effects such as weight gain and affected cholesterol, triglyceride, glucose, and/or other blood lipid levels due to faster dissolving and absorption, among other factors. In addition, sublingual delivery may allow lower dosages to be used.

As still another example, the active ingredients in some embodiments may include lurasidone. Lurasidone is an antipsychotic medication that may be used to treat conditions such as schizophrenia and bipolar disorder, among others. Lurasidone may be associated with side effects including nausea and minor sedation. Lurasidone may be used in SDV dosages such as, for example, ten milligrams, twenty milligrams, forty milligrams, and sixty milligrams, where such dosages may be half the dosage of current oral pills. Lurasidone may be taken in a single dose per day.

Lurasidone tablets may be provided in dosages ranging from ten milligrams to sixty milligrams. Sublingual delivery of lurasidone is associated with reduction or elimination of side effects such as nausea, minor sedation, weight gain, and lipid and/or glucose elevations. Oral pills currently require lurasidone to be taken with at least a three hundred fifty calorie meal. Such a requirement reduced compliance, as users do not want to eat a large meal near bedtime, nor do the users want to take the medication earlier as they may be drowsy, fall asleep too early, or otherwise not be able to perform various tasks (e.g., driving).

In contrast to oral pills, the SDVs of some embodiments allow users to receive a lurasidone dose that bypasses the gastrointestinal tract and thus does not require taking lurasidone with a meal, thus also allowing users to take the medication closer to bedtime. Further, by bypassing first-pass metabolism, the SDVs of some embodiments minimize drug and/or food interactions through hepatic metabolism. Such SDVs may lessen or eliminate label warnings, such as for the avoidance of grapefruit or grapefruit juice, medication that induce or inhibit the hepatic metabolism (e.g., CYP 3A4 enzyme).

As still another example, the active ingredients in some embodiments may include vortioxetine. Vortioxetine is a medication that may be used to treat conditions such as major depressive disorder, among others. Vortioxetine may be associated with side effects including nausea, diarrhea, and constipation. Vortioxetine may be used in SDV dosages ranging from, for example, two-and-a-half milligrams to ten milligrams, where such dosages may be half the dosage of current oral pills. Vortioxetine may be taken in a single dose per day.

Vortioxetine tablets may be provided in dosages ranging from two-and-a-half milligrams to ten milligrams. Sublingual delivery of vortioxetine is associated with reduction or elimination of gastrointestinal side effects such as nausea, diarrhea, and constipation. Further, by bypassing first-pass metabolism, the SDVs of some embodiments minimize drug and/or food interactions through hepatic metabolism (e.g., CYP 2D6 enzyme).

As still another example, the active ingredients in some embodiments may include brexpiprazole. Brexpiprazole is an atypical antipsychotic medication that may be used to treat conditions such as schizophrenia, and as an adjunctive treatment for depression, among others. Brexpiprazole may be associated with side effects including weight gain. Brexpiprazole may affect cholesterol and triglyceride levels, and glucose and other blood lipid levels, among others. Brexpiprazole may be used in SDV dosages ranging from, for example, one quarter milligram to two milligrams, where such dosages may be half the dosage of current oral pills. Brexpiprazole may be taken in a single dose per day.

Brexpiprazole SDV tablets may be provided in dosages such as one quarter milligram, one half milligram, one milligram, one-and-a-half milligrams, and two milligrams. Sublingual delivery of brexpiprazole is associated with reduction or elimination of side effects such as weight gain and affected cholesterol, triglyceride, glucose, and/or other blood lipid levels due to faster dissolving and absorption, among other factors. Further, by bypassing first-pass metabolism, the SDVs of some embodiments minimize drug and/or food interactions through hepatic metabolism. Such SDVs may lessen or eliminate label warnings, such as for the avoidance of grapefruit or grapefruit juice, medication that induce or inhibit the hepatic metabolism (e.g., CYP 3A4 enzyme, CYP 2D6 enzyme, etc.).

Furthermore, sublingual delivery allows quicker absorption that results in more rapid effect when used to treat schizophrenia, bipolar disorder, stuttering, Tourette Syndrome, or major depressive disorder. Faster elimination may also reduce side effects associated with blonanserin use.

One of ordinary skill in the art will recognize that although various examples above and below may describe specific SDVs, active ingredients, inactive ingredients, etc., various other SDVs, active ingredients, inactive ingredients, etc. may be included, utilized, or implemented by some embodiments. For instance, although some ingredients may be described by reference to materials included in a tablet SDV, the same or similar ingredients may be included in a capsule, gel strip, or other type of SDV. Further, although various compounds may be described by reference to particular conditions, the same or similar compounds may be used to treat various other conditions. As another example, although attributes may be specified for a particular SDV (e.g., a dissolving time of thirty seconds for a sublingual tablet), such attributes may be similar or the same for other types of SDVs (e.g., a gel strip may also dissolve within thirty seconds).

Many chemical entities, compounds, and families have been profiled, and research has demonstrated unexpected benefits of delivering them sublingually. Accordingly, lower dosages of select compounds have achieved unexpectedly better results when delivered sublingually.

Among those moieties best served by sublingual approaches to bioavailability improvements are exemplary compounds and other common agents used for treatment of pulmonary hypertension (e.g., phosphodiesterase-5 (PDE-5) inhibitors), high blood pressure, cholesterol issues, vasodilation, and diabetes, among others.

Generally, sublingual dosage forms dissolve within a time period of at least about two minutes, but less than about seven minutes. Dissolving time in water for the presently contemplated dosage forms ranges from about three minutes to about five minutes.

Formulations including an active agent, such as insulin, and one or more excipients, such as a chelator and/or solubilizing agent, may dissolve rapidly in aqueous medias. In select embodiments, the formulations are suitable for subcutaneous or sublingual administration. These formulations are rapidly absorbed through mucosal surfaces (parenteral, pulmonary, etc.) and through the fatty tissue when administered subcutaneously. Such absorption is achieved through the addition of excipients, especially solubilizers such as acids and metal chelators.

As generally used herein, a drug is considered “highly soluble” when the highest dose strength is soluble in two hundred fifty milliliters or less of aqueous media over the pH range of 1-7.5. The volume estimate of two hundred fifty milliliters is derived from typical bioequivalence (BE) study protocols that prescribe administration of a drug product to fasting human volunteers with a glass (about eight ounces) of water. A drug is considered highly soluble when ninety percent or more of an administered dose, based on a mass determination or in comparison to an intravenous reference dose, is dissolved. Solubility can be measured by the shake-flask or titration method or analysis by a validated stability-indicating assay.

As generally used herein, an immediate release drug formulation is considered “rapidly dissolving” when no less than eighty-five percent of the labeled amount of the drug substance dissolves within thirty minutes, using U.S. Pharmacopeia (USP) Apparatus I at one hundred rpm (or Apparatus II at fifty rpm) in a volume of nine hundred milliliters or less in each of the following media: (1) 0.1 N HCI or Simulated Gastric Fluid USP without enzymes; (2) a pH 4.5 buffer; and (3) a pH 6.8 buffer or Simulated Intestinal Fluid USP without enzymes.

Although described with reference to small-molecule drugs like insulin, the instant formulations may be used with other agents, including peptides, proteins, nucleotide molecules (RNA sequences, DNA sequences), sugars, polysaccharides, and small organic molecules. In some examples, the active agent is at least slightly soluble in aqueous medium (e.g., ten thousand parts of aqueous solvent per solute), and in others, is highly soluble in aqueous medium. Preferably the active agent is highly potent, so that only a small amount (e.g., in the microgram range) is needed to provide a therapeutic effect. Suitable peptides include but are not limited to insulin and derivatives of insulin, such as lispro; C-peptide; glucagon-like peptide 1 (GLP 1) and all active fragments thereof; human amyl in and synthetic forms of amyl in, such as pramlintide; parathyroid hormone (PTH) and active fragments thereof (e.g., PTH1-34); calcitonin; human growth hormone (HGH); erythropoietin (EPO); macrophage-colony stimulating factor (M-CSF); granulocyte macrophage colony stimulating factor (GM-CSF); and interleukins. In the preferred embodiment the active agent is insulin. Suitable small molecules include nitroglycerin, sumatriptan, narcotics (e.g., fentanyl, codeine, propoxyphene, hydrocodone, and oxycodone), benzodiazepines (e.g. alprazolam, clobazam, clonazepam, diazepam flunitrazepam, lorazepam, nitrazepam, oxazepam, temazepam, and triazolam), phenothiazines (chlorpromazine, fluphenazine, mesoridazine, methotrimeprazine, pericyazine, perphenazine, prochlorperazine, thioproperazine, thioridazine, and trifluoperazine), and selective serotonin reuptake inhibitors (SSRIs) (e.g., sertraline, fluvoxamine, fluoxetine, citalopram, and paroxetine).

The dosages of the active agents depend on their bioavailability and the condition, ailment, disease or disorder to be treated. The compositions optionally include one or more excipients.

In select embodiments, one or more solubilizing agents are included with the active agent to promote rapid dissolution in aqueous media. Suitable solubilizing agents include wetting agents such as polysorbates and poloxamers, non-ionic and ionic surfactants, food acids and bases (e.g., sodium bicarbonate), and alcohols, and buffer salts for pH control. Suitable acids include acetic acid, ascorbic acid, citric acid, and hydrochloric acid. For example, if the active agent is insulin, a preferred solubilizing agent is citric acid, as known to those skilled in the art.

Diluents, also referred to herein as fillers, are typically necessary to increase the bulk of a solid dosage form so that a practical size is provided for compression of tablets or formation of beads and granules. Suitable fillers include, but are not limited to, dicalcium phosphate dihydrate, calcium sulfate, lactose, sucrose, mannitol, sorbitol, cellulose, microcrystalline cellulose, powdered cellulose, kaolin, sodium chloride, dry starch, hydrolyzed starches, pregelatinized starch, silicone dioxide, titanium oxide, magnesium aluminum silicate, calcium carbonate, compressible sugar, sugar spheres, powdered (confectioner's) sugar, dextrates, dextrin, dextrose, dibasic calcium phosphate dehydrate, glyceryl palmitostearate, magnesium carbonate, magnesium oxide, maltodextrin, polymethacrylates, potassium chloride, talc, and tribasic calcium phosphate.

Binders are used to impart cohesive qualities to a solid dosage formulation, and thus ensure that a tablet, bead or granule remains intact after the formation of the dosage forms. Suitable binder materials include, but are not limited to, starch, pregelatinized starch, gelatin, sugars (including sucrose, glucose, dextrose, lactose and sorbitol), dextrin, maltodextrin, zein, polyethylene glycol, waxes, natural and synthetic gums such as acacia, guar gum, tragacanth, alginate, sodium alginate, celluloses, including hydroxypropyl methylcellulose, carboxymethylcellulose (CMC) sodium, hydroxypropyl cellulose, hydroxyethyl cellulose, ethyl cellulose, methyl cellulose, and smectite, hydrogenated vegetable oil, Type I, magnesium aluminum silicate, and synthetic polymers such as acrylic acid and methacrylic acid copolymers, carbomer, methacrylic acid copolymers, methyl methacrylate copolymers, aminoalkyl methacrylate copolymers, polyacrylic acid, polymethacrylic acid, and polyvinylpyrrolidone.

Lubricants are used to facilitate tablet manufacture. Examples of suitable lubricants include, but are not limited to, magnesium stearate, calcium stearate, stearic acid, glyceryl behenate, glyceryl mono stearate, glyceryl palmitostearate, hydrogenated castor oil, hydrogenated vegetable oil, type I, sodium benzoate, sodium lauryl sulfate, sodium stearyl fumarate, polyethylene glycol, talc, zinc stearate, and mineral oil and light mineral oil.

Stabilizers are used to inhibit or retard drug decomposition reactions which include, by way of example, oxidative reactions. A number of stabilizers may be used.

Surfactants may be anionic, cationic, amphoteric or nonionic surface-active agents. Suitable anionic surfactants include, but are not limited to, those including carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate.

If desired, the tablets, wafers, gel strips, films, lozenges, beads, granules, particles, and/or other SDVs may include a specified amount of nontoxic auxiliary substances such as dyes, masking agents, sweeteners, coloring and flavoring agents, pH buffering agents, or preservatives.

Blending or copolymerization sufficient to provide a certain amount of hydrophilic character can be useful to improve wettability of the materials. The active compounds (or pharmaceutically acceptable salts thereof) may be administered in the form of a pharmaceutical composition wherein the active compound(s) is in admixture or mixture with one or more pharmaceutically acceptable carriers, excipients or diluents. Suitable dosage forms include powders, films, wafers, lozenges, capsules, and tablets. Following administration, the dosage form dissolves quickly releasing the drug or forming small particles including the drug, optionally including one or more excipients.

Select variations of the various formulations described herein may dissolve in a time period ranging from one second to at least about three minutes, three to five minutes, five to eight minutes, or eight to twelve minutes. In some embodiments, dissolving time is less than thirty seconds. According to the instant teachings, the drugs are absorbed and transported to the plasma quickly, resulting in a rapid onset of action (for example, beginning within about five minutes following administration and peaking at about fifteen to thirty minutes following administration).

By way of further example of the benefits of the instant teachings as applied to treating pulmonary hypertension, extremely low dosages of compounds like sildenafil can be efficacious, have lower risk profiles, and may have other and further advantages when delivered with all-natural vehicles and systems.

It is known that oral medicines are particularly desirable and sought-after discreet form of treatment for sexual dysfunction. Recently, the oral use of the citrate salt of sildenafil has been approved by the U.S. Food and Drug Administration (FDA) for the treatment of male erectile dysfunction. Sildenafil is reported to be a selective inhibitor of cyclic-guanosine monophosphate (GMP)-specific phosphodiesterase type 5 (PDES), the predominant isozyme metabolizing cyclic GMP formed in the corpus cavernosum. Because sildenafil is a potent inhibitor of PDES in the corpus cavernosum, it is believed to enhance the effect of nitric oxide release. Inasmuch as sildenafil at the currently recommended doses of twenty-five to one hundred milligrams has little effect in the absence of sexual stimulation, sildenafil is believed to restore the natural erectile response to sexual stimulation but not cause erections in the absence of such stimulation. The localized mechanism by which cyclic GMP stimulates relaxation of the smooth muscles has not been elucidated.

In dose-response studies, increasing doses of sildenafil (twenty-five to one hundred milligrams) reportedly increased the erectogenic efficacy of sildenafil. However, the oral administration of sildenafil is also accompanied by dose-responsive undesirable side effects, including more serious side effects, such as syncope (loss of consciousness), priapism (erection lasting four hours or more) and increased cardiac risk (coital coronaries). It is noted these can be brought on in some cases by physiological predisposition, adverse drug interaction or potentiation, or by drug abuse. In particular, hypotension crisis can result from the combination of sildenafil citrate and organic nitrates, causing, in some cases death, so its administration to patients who are concurrently using organic nitrates (such as nitroglycerin) in any form is contraindicated. Thus, there is a need and desire for oral administration forms that promote the bioavailability of sildenafil at lower doses while minimizing side effects.

Early-stage sublingual tablets are well documented in the literature since the beginning of this century. The main reason for sublingual route of drug administration is to provide a rapid onset of action of potent drugs. Another reason is to avoid the first pass metabolism by the liver.

The term “controlled release” when applied to sublingual tablets is limited to a maximum of about sixty minutes. Traditional sublingual tablets are usually designed as water soluble tablets made of water-soluble sugars such as sorbitol, lactose, mannitol, etc. In the literature, controlled release sublingual tablets are very scarce. Active ingredients such as nitroglycerin, caffeine, guaiocolate, amylase or isoproterenol were then added to the pourable paste that was cast into tablets. Such techniques are not appropriate to make tablets by compression. The time of release for a pharmaceutical preparation is critical to the effectiveness of the drug. The sublingual tablet of the present invention can be prepared by compression methods and provides a controlled drug release, in contradistinction to the prior art.

Therefore, the sildenafil-analogues including sildenafil, homosildenafil, hydroxyhomosildenafil, desmethylsildenafil, acetildenafil, vardenafil and udenafil, are interesting given the delivery system of the instant teachings. The sildenafil may represent those seven compounds, may react with statin derivative, y-polyglutamic acid derivative, vitamin or sodium CMC to form the monoquaternary amine complex salts of Sildenafil-analogues and amine complex salts of udenafil-analogues. Thereby, sildenafil-analogues may represent sildenafil, homosildenafil, hydroxyhomosildenafil, desmethylsildenafil, acetildenafil, vardenafil and udenafil. The involved piperazine or amine moiety, and the statins, y-polyglutamic acid derivative, vitamin or sodium CMC may represent ostensive or potential combinations effective for sublingual delivery in accordance with the instant teachings.

Thus, the lactone ring, ester and protected derivatives of the Statins are available to prepare the above sildenafil-analogues monoquaternary amine complex salts or udenafil-analogues amine complex salts deliverable according to the instant teachings.

Likewise, statins derivative and y-polyglutamic acid derivative, Vitamin or sodium CMC separately react with the piperazine group of Sildenafil-analogues or pyrrolidinyl group of sildenafil-analogues to prepare the sildenafil-analogues monoquaternary complex salts or sildenafil analogues amine complex salts. Preferred statin derivatives are selected from atorvastatin, lovastatin, pitavastatin, rosuvastatin and simvastatin, y-polyglutamic acid derivative are selected from alginate sodium, the y-polyglutamic acid, the sodium polyglutamate, and the glutamine transporter (GLT) is referred as the copolymer of lysine, glutamate and tyrosine, and the calcium polyglutamate-alginate sodium, vitamin is selected from retinoic acid, ascorbic acid, folic acid, gamma-linolenic acid, nicotinic acid and pantothenic acid. Thereby, the sildenafils-y-polyglutamic acid, sildenafils-simvastatinic acid, sildenafils-pramastatinic acid, sildenafils-lovastatinic acid, sildenafils-pitavastatin, sildenafils-rosuvastatin sildenafil-L-arginine, sildenafil-CMC, sildenafil-mevastatinic acid, sildenafil-rosuvastatinic acid, sildenafils-lovastatinic acid, udenafil-CMC, udenafil-nicotinic acid and udenafil-L-retinoic acid are obtained.

The term excipients or “pharmaceutically acceptable carrier or excipients” and “bio-available carriers or excipients” above-mentioned include any appropriate compounds known to be used for preparing the dosage form, such as the solvent, the dispersing agent, the coating, an anti-bacterial or anti-fungal agent and a preserving agent or the delayed absorbent. Usually, such kind of carrier or excipient does not have therapeutic activity itself. Each formulation prepared by combining the derivatives disclosed in the present invention and the pharmaceutically acceptable carriers or excipients will not cause the undesired effect, allergy or other inappropriate effects while being administered to an animal or human. Accordingly, the derivatives disclosed in the present invention in combination with the pharmaceutically acceptable carrier or excipients are adaptable in the clinical usage and in the human. A therapeutic effect can be achieved by using the 03 dosage form in the present invention by sublingual administration. About one hundred micrograms to ten milligrams per day of the active ingredient is administered for the patients of various diseases.

Possible agents to be combined include statins selected from a group including atorvastatin, lovastatin, pitavastatin, rosuvastatin and simvastatin, and the statin structure of those drugs are hydrolyzed by metallic hydroxide, such as sodium, potassium, calcium, and ammonia hydroxide, and acids useful to hydrolyze the ester group of statins.

The formation of sildenafils-statinic acid complex from sildenafils HCI salt is easily obtained by reacting sildenafils HCI with the equal molar sodium hydroxide in the presence of hydrolyzable Statins or Statins ester and derivatives. The sodium ion precedes the equal molar neutralization can take place within the HCI part of sildenafils HCI, and the resulted NaCl is dissolved in the hydrated alcohol solution. The statin shows the ionic state, the free state or being mixed with other unreacted ester derivative of the statin in a mixing solution of water and C1-C4 lower alcohol (i.e., the ethanol and the isopropanol). By following the amount of each Statin derivative hydrolyzed by the sufficient amount of sodium hydroxide, the term “sufficient amount of piperazium group or pyrrolidinyl group” is about the amount of equal mole.

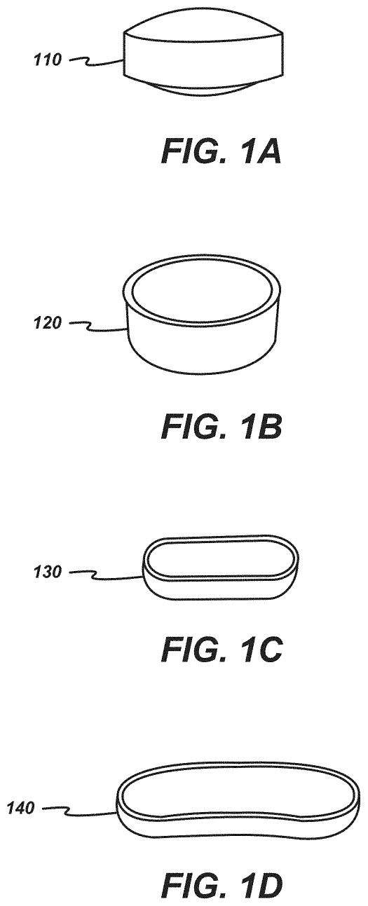

FIG. 1A illustrates an example of one or more embodiments described herein, in which a sublingual tablet 110 has a round convex shape. FIG. 1B illustrates an example of one or more embodiments described herein, in which a sublingual tablet 120 has a round concave shape. FIG. 1C illustrates an example of one or more embodiments described herein, in which a sublingual tablet 130 has an oval concave shape. FIG. 1D illustrates an example of one or more embodiments described herein, in which a sublingual tablet 140 has a curved oval concave shape.

The thicker body of the round convex sublingual tablet 110 slows dissolution while the convex shape causes movement under the tongue. The shape of the round concave sublingual tablet 120 enables saliva to pool in order to speed dissolvability along with providing a modicum of suction in order to reduce movement.

The shape of the oval concave sublingual tablet 130 provides a dish-like recess which pools saliva in order to speed dissolvability. The elongated shape of the oval concave sublingual tablet 130 reduces movement under the tongue. The elongated shape of the curved oval concave sublingual tablet 140 may improve functional engagement of the user's tongue to reduce movement and may also provide a recess that pools saliva in order to speed dissolvability. Further, the thickness of the curved oval concave sublingual tablet 140 may be less than other shapes, promoting faster dissolving and reduced movement.

Each of the sublingual tablets 110-140 may include compressed dry powder and/or other appropriate ingredients. The sublingual tablets 110-140 may be size adjusted such that each tablet includes the same volume of dry powder and/or other appropriate ingredients.

Different embodiments may include various differently shaped tablets than shown, such as squares, rectangles, almond-shaped, pentagons, triangle, core rod, oval, lozenge, etc.). In addition, different embodiments may include differently sized tablets. For instance, the diameter or thickness of round convex tablets 110 and/or round concave tablets 120 may be varied based on various relevant factors (e.g., amount of dose, desired dissolving time, age or capacity of patient, etc.). As another example, the size and/or contours of the recesses of the oval concave sublingual tablet 130 and/or the curved oval concave sublingual tablet 140 may be various based on relevant factors such as desired dissolving time).

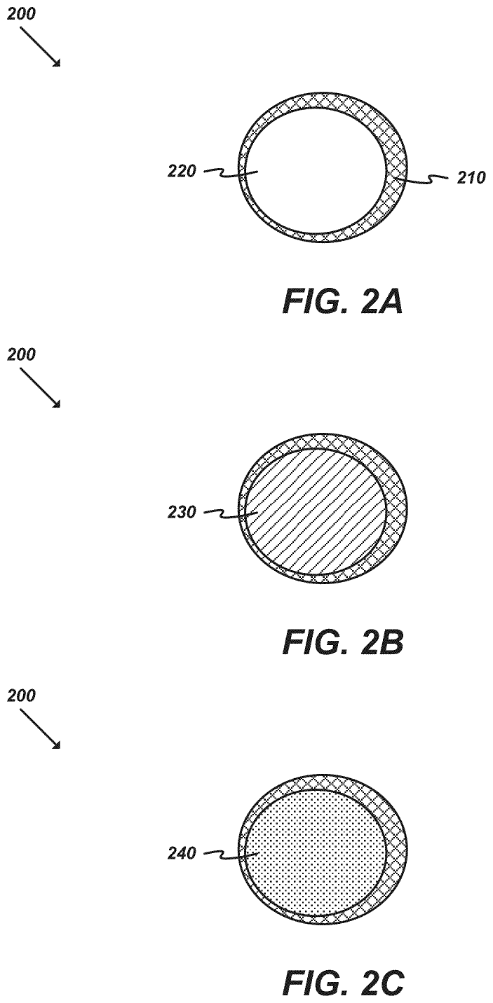

FIG. 2A illustrates an example of one or more embodiments described herein, in which a sublingual capsule 200 includes a casing 210 and interior filling 220. FIG. 2B illustrates an example of one or more embodiments described herein, in which a sublingual capsule 200 includes a liquid or gel filling 230. FIG. 2C illustrates an example of one or more embodiments described herein, in which a sublingual capsule 200 includes a dry powder filling 240.

The capsules 200 may be extruded in some embodiments. The casing 210 may include an eccentric gelatin capsule casing. The filling extrusion 220 may include various ingredients and may be extruded in various different forms, such as liquid or gel filling 230 and/or dry powder filling 240. Liquid and/or gel ingredients may be extruded in interior fillings 230. The terms “gel” or “gelatin” may be used to refer to substances or compounds that include gelatin and/or other thickening agents, liquids, etc. Dry powder ingredients 240 may be blown into the center cavity of the casing 210.

The capsules 200 may be manufactured using a continuous extrusion process whereby an eccentric gelatin capsule casing, such as casing 210, may be extruded, a gelatin plug (not shown) may be extruded, an interior filling 220 (e.g., liquid or gel filling 230 and/or dry powder filling 240) may be extruded, and a gelatin plug may be extruded to seal the capsule 200.

The diameter of the capsules 200 may be set by an extrusion die. The capsule 200 may be cut to a desired length. Such eccentric capsules 200 may include a thin wall or casing 210 to aid dissolving without additional processes.

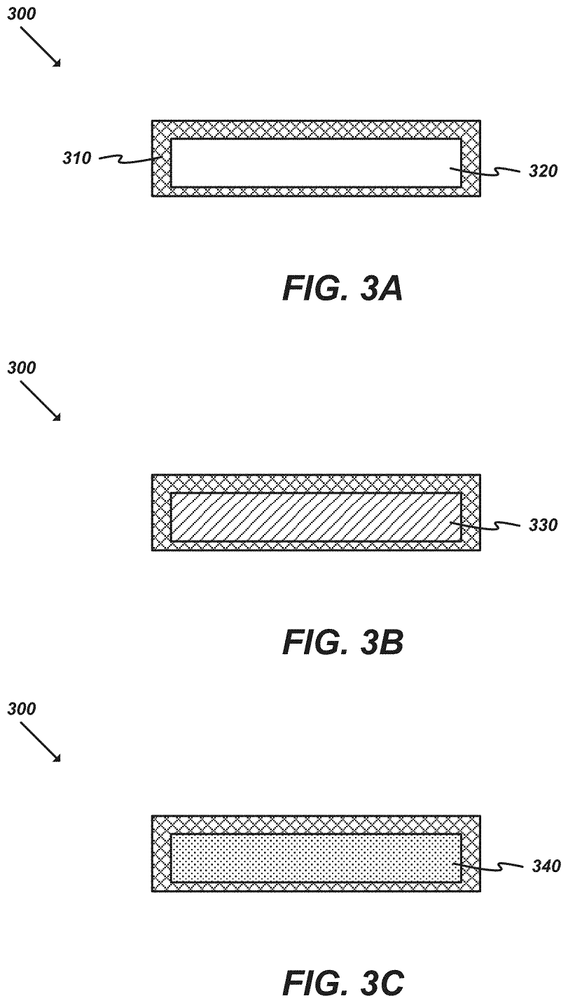

FIG. 3A illustrates an example of one or more embodiments described herein, in which an offset extruded sublingual strip 300 includes a casing 310 and extruded filling 320. FIG. 3B illustrates an example of one or more embodiments described herein, in which an offset extruded sublingual strip 300 includes a liquid or gel filling 330. FIG. 3C illustrates an example of one or more embodiments described herein, in which an offset extruded sublingual strip 300 includes a dry powder filling 340.

The offset extruded gel strip 300 may be manufactured using a continuous extrusion process whereby an offset extruded gelatin strip casing, such as casing 310, may be extruded, a gelatin plug (not shown) may be extruded, an interior filling 320 may be extruded, and another gelatin plug may be extruded to seal the capsule.

The dimensions of the offset extruded sublingual strip 300 may be set by an extrusion die. The strip 300 may be cut to a desired length. Such strips 300 may include a thin wall to aid dissolving without additional processes.

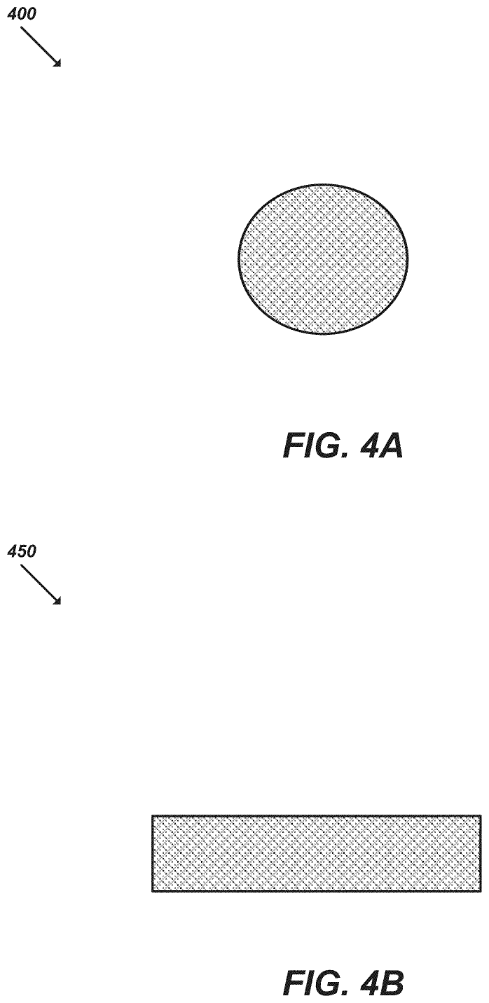

FIG. 4A illustrates an example of one or more embodiments described herein, in which a sublingual capsule extrusion 400 is generated. FIG. 4B illustrates an example of one or more embodiments described herein, in which a sublingual strip extrusion is generated 450.

Dry powder ingredients, such a dry powder ingredients 240 or dry powder filling 340 may be mixed with gelatin (and/or other appropriate thickening agents) and extruded together with a casing such as casing 210 or casing 310. In some embodiments, a substance such as edible food starch may be dusted onto the exterior surfaces of the capsule extrusion 400 or strip extrusion 450 to prevent product from sticking together. The capsule extrusion 400 may have a generally cylindrical shape and may be cut to a desired length (e.g., to achieve a specified dosage) after extrusion. The gel strip extrusion 450 may have, for example, an elongated cube shape and may be cut to a desired length after extrusion.

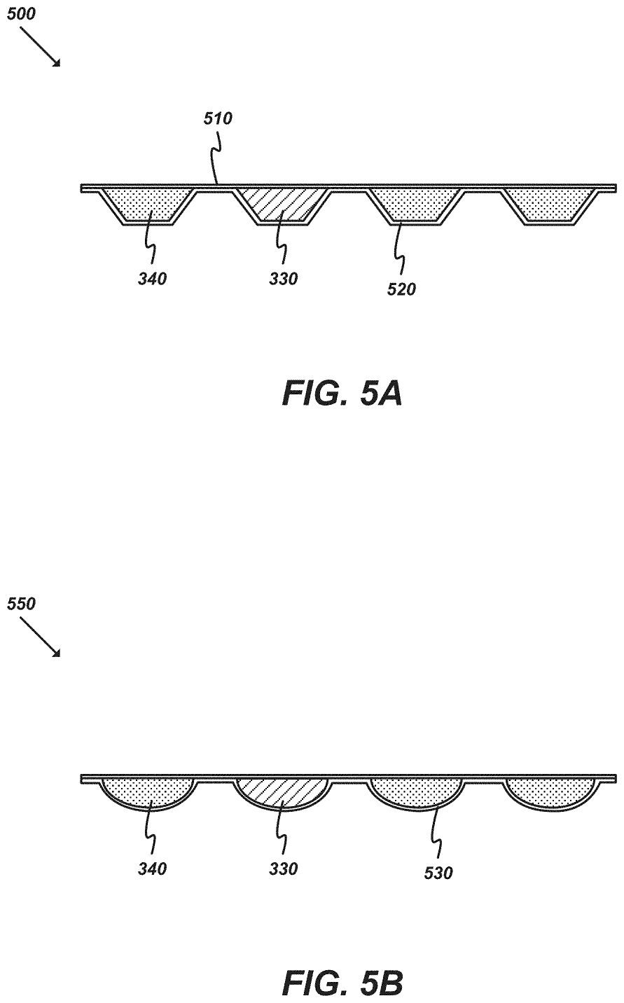

FIG. 5A illustrates an example of one or more embodiments described herein, in which waffle gel strips 500 include ingredient fillings 330-340. FIG. 5B illustrates an example of one or more embodiments described herein, in which dimpled gel strips 550 include ingredient fillings 330-340.

Such waffle gel strips 500 and dimpled gel strips 550 may be manufactured using a non-extruded process whereby continuous manufacture is provided via a mold and imprint wheel. Such an approach is easily expandable to produce multiple product lines simultaneously. The molds and imprint wheel may include materials such as silicone. Each strip may include multiple ingredient combinations as cell fillings. Ingredient fillings may include dry powders, liquid or mixed gelatin fills, and/or other appropriate fills.

In some embodiments, the waffle gel strips 500 and/or dimple gel strips 550 may include a textured surface, such as surface 510 to reduce movement under the tongue. The bottom of each cell structure 520 or 530 may be thinned such that fast dissolving and release of ingredients is promoted.

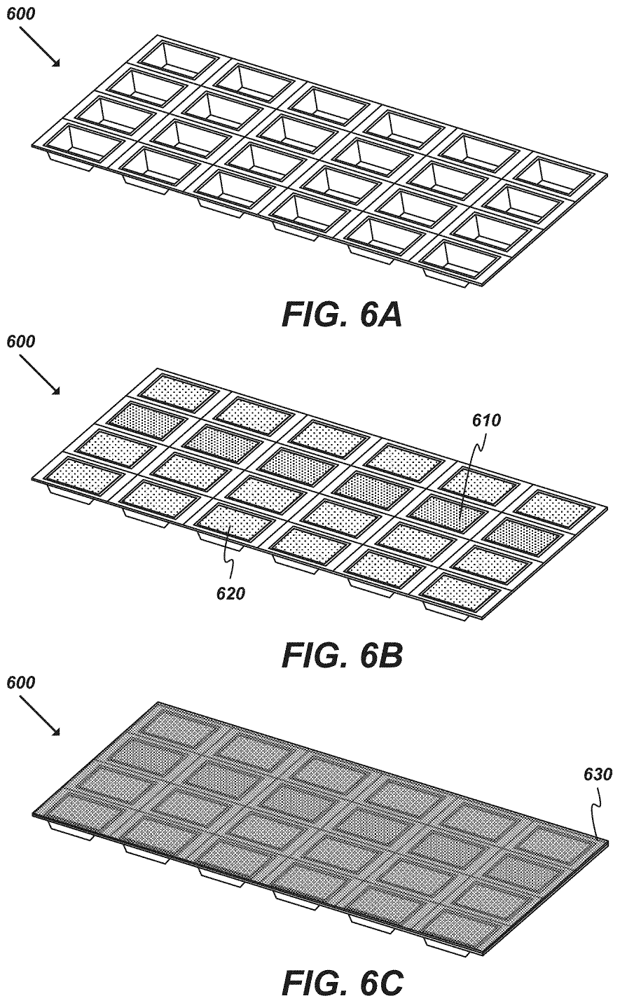

FIG. 6A illustrates an example of one or more embodiments described herein, in which a sheet 600 of uncut waffle gel strips, including cells such as those included in gel strips 500 or 550 is ready to be filled. FIG. 6B illustrates an example of one or more embodiments described herein, in which a sheet 600 of uncut waffle gel strips includes fillings such as liquid fillings 610, dry powder fillings 620, and/or mixed fillings (e.g., dry powder mixed with gelatin). FIG. 6C illustrates an example of one or more embodiments described herein, in which a sheet 600 of uncut waffle gel strips includes a gel cover 630.

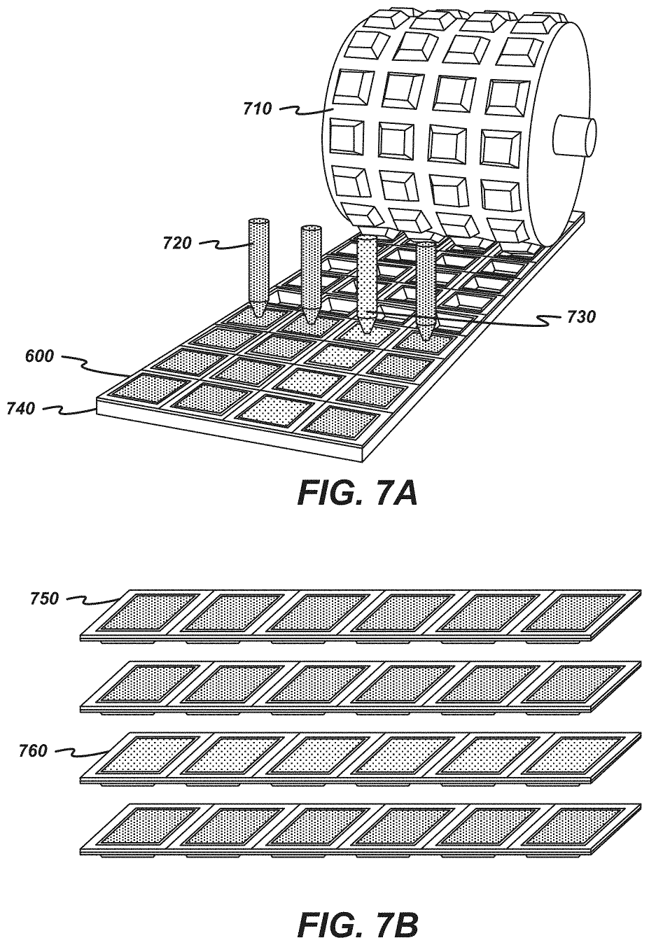

FIG. 7A illustrates an example of one or more embodiments described herein, in which a sheet of waffle gel strips, such as sheet 600, is imprinted and filled. As shown, a waffle thinning imprint wheel 710 may be used to generate sheet 600. The individual cells may be filled using ingredient filling nozzles 720-730, where fillings may include any combination of dry powders, liquids, gel mixtures, etc. In this example, ingredient filling nozzles 720 that dispense a first ingredient filling are indicated by a first fill pattern, while ingredient filling nozzles 730 that dispense a second ingredient filling are indicated by a second fill pattern. In this example, each row (or column) of the imprinted sheet 600 may include a single ingredient filling. Different embodiments may dispense different combinations of ingredient fillings in various different ways (e.g., by including multiple nozzles for each row or column, using movable nozzles, etc.). The imprint wheel 710 may be used with a complementary base 740 or similar element that retains the sheet 600 and/or guides the imprint wheel 710.

FIG. 7B illustrates an example of one or more embodiments described herein, in which a sheet 600 of waffle gel strips has been cut into filled strips 750-760. In this example, gel strips 750 including a first ingredient filling are indicated by a first fill pattern, while gel strips 760 including a second ingredient filling are indicated by a second fill pattern. As discussed above, different embodiments may include various different combinations of fillings in each strip. In this example, each gel strip 760 includes six cells arranged in a single row or column, but different embodiments may include various different numbers and/or arrangements of cells.

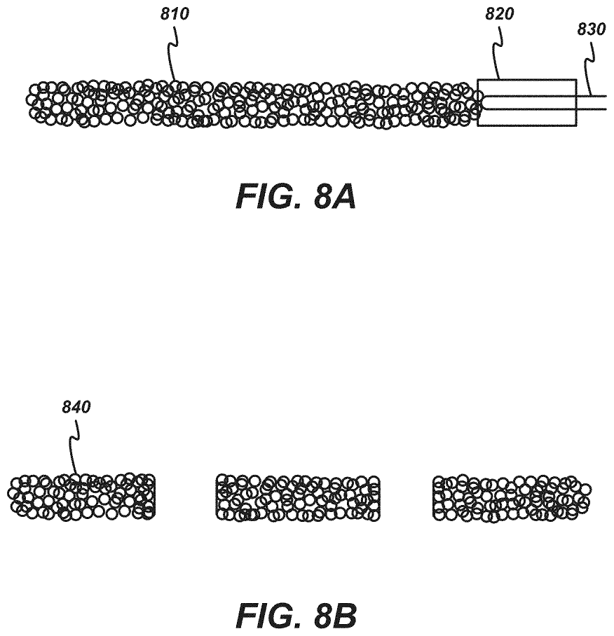

FIG. 8A illustrates an example of one or more embodiments described herein, in which a bubbled gel mixture strip rope 810 is generated. The gel mixture strip rope 810 may be generated using a gelatin-ingredient mixture supply 820 and a compressed air injection supply 830. FIG. 8B illustrates an example of one or more embodiments described herein, in which a bubbled gel mixture strip rope 810 is has been cut into strips 840.

As shown, sublingual bubbled gel strips 840 with a gelatin-ingredient mixture 820 may be generated using a continuous compressed air injection 830 into the gelatin-ingredient mixture 820. Such a process is easily expandable to produce multiple product lines simultaneously. The diameter of the bubbled strip rope 810 may be configured based on the flow and/or volume of gelatin-ingredient mixture 820, air or ingredient pressure, tip type associated with supply nozzles, and/or other relevant attributes or operating parameters (e.g., tip size). Each rope 810 may include multiple ingredient combinations mixed with gelatin. The ingredient fillings may include dry powders or liquids mixed with gelatin.

The gel strip 840 provides a textured surface that reduces movement under the tongue. The bubbled structure thickness may be controlled by air injection such that a thin structure may be achieved, resulting in a gel strip 840 that dissolves quickly.

A faster dissolving time may have several benefits, including reducing the time a user must refrain from eating or drinking, reducing the time that speech is impeded by presence of the gel strip 840 (and/or other SDV), and promoting faster release and absorption of ingredients.

Throughout this disclosure, the term “sublingual products” may be used to refer to any, some, or all of the various examples described above in reference to FIG. 1A-FIG. 8B.

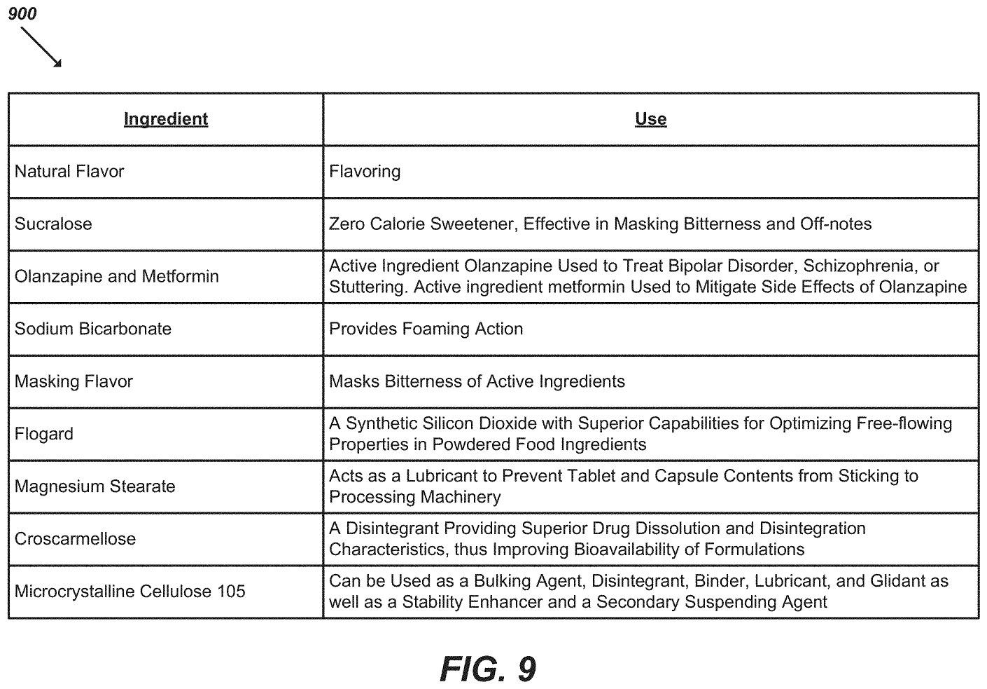

FIG. 9 illustrates an example of one or more embodiments described herein, in which an ingredient listing 900 is provided for an improved formulation that includes olanzapine and metformin as active ingredients. Such a formulation allows dosage requirements to be stepped down, as well as overcoming bitterness and/or gustatory issues. Further, the combination may reduce side effects associated with olanzapine.

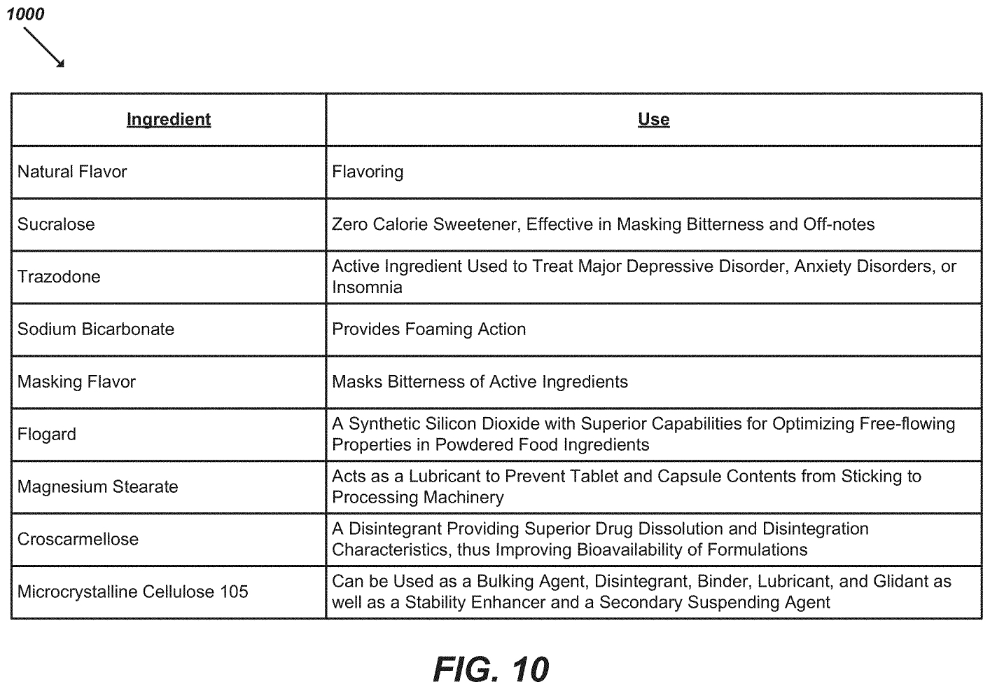

FIG. 10 illustrates an example of one or more embodiments described herein, in which an ingredient listing 1000 is provided for an improved formulation that includes trazodone as an active ingredient. Such a formulation allows dosage requirements to be stepped down, side effects to be mitigated, as well as overcoming bitterness and/or gustatory issues.

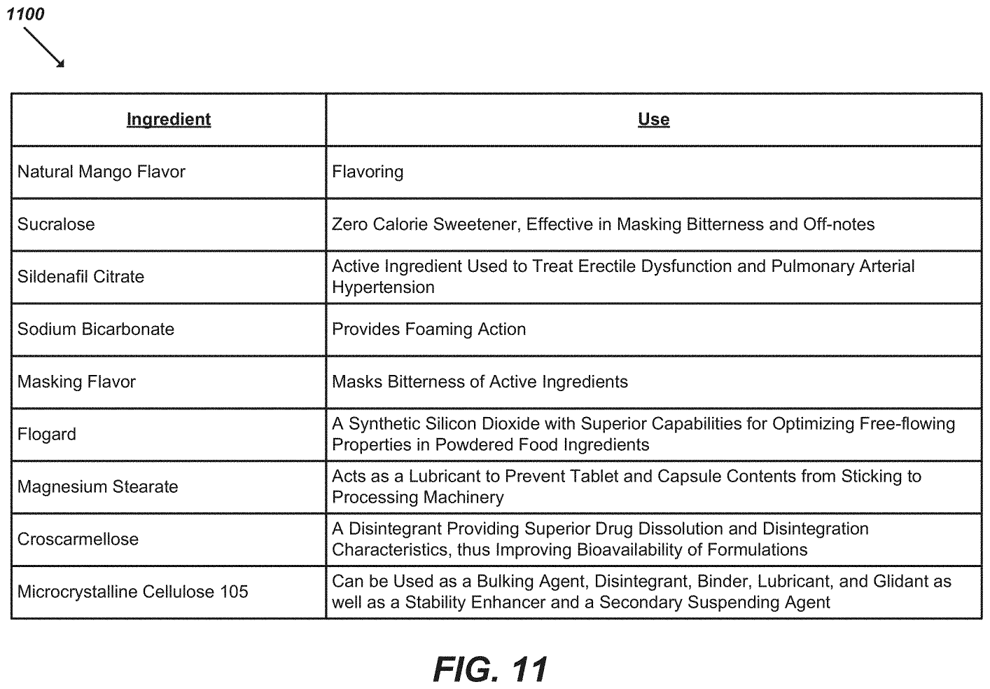

FIG. 11 illustrates an example of one or more embodiments described herein, in which an ingredient listing 1100 is provided for an improved formulation that includes sildenafil citrate as an active ingredient. Such a formulation allows dosage requirements to be stepped down, as well as overcoming bitterness and/or gustatory issues.

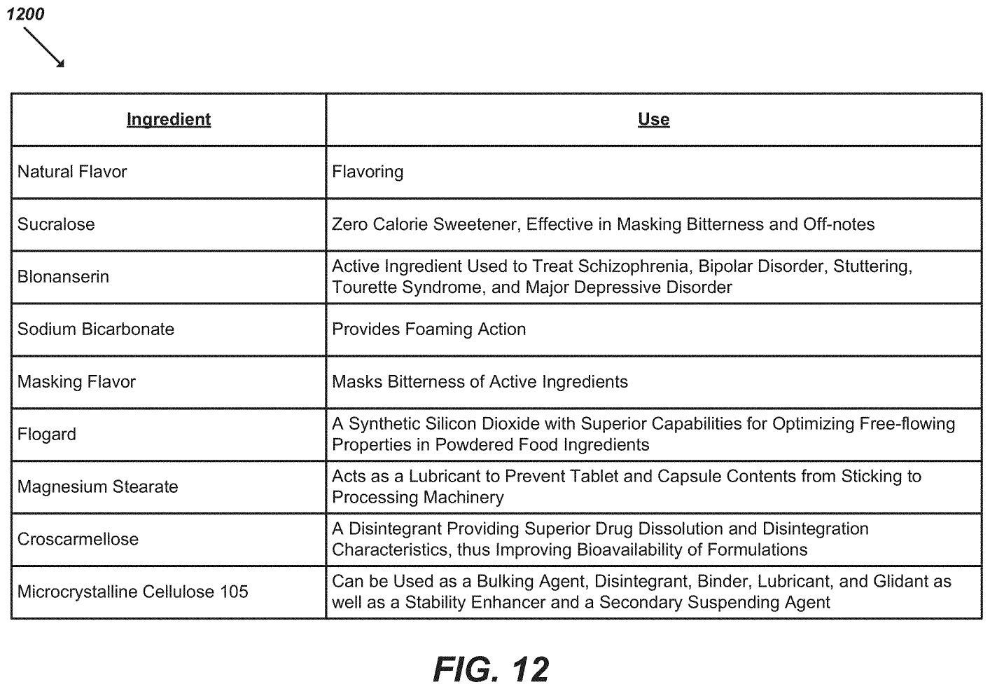

FIG. 12 illustrates an example of one or more embodiments described herein, in which an ingredient listing 1200 is provided for an improved formulation that includes blonanserin as an active ingredient. Such a formulation allows dosage requirements to be stepped down, side effects to be mitigated, as well as overcoming bitterness and/or gustatory issues.

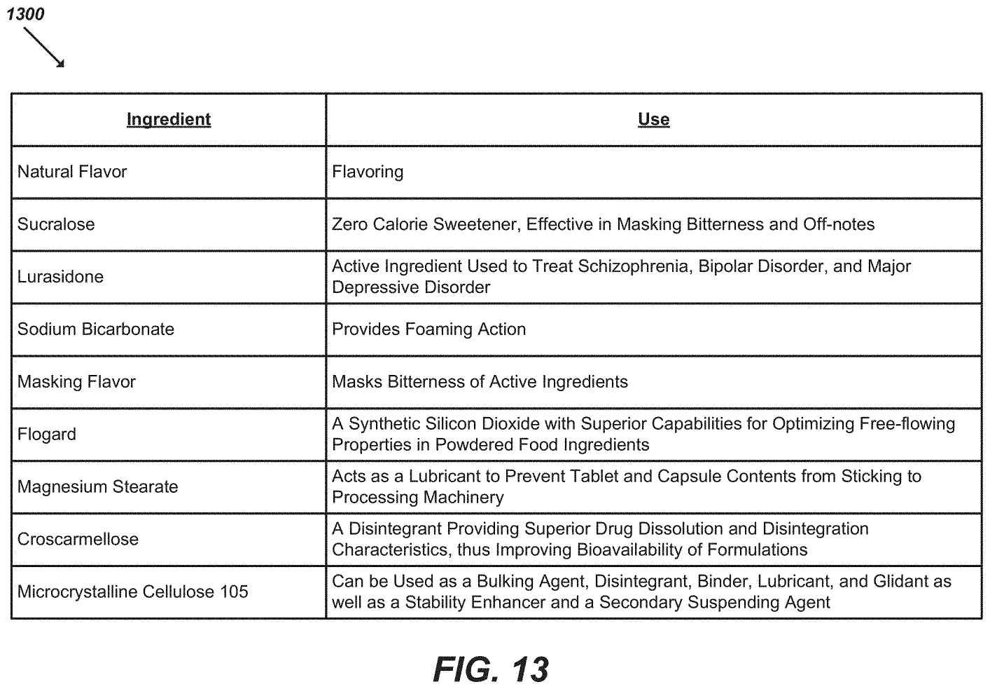

FIG. 13 illustrates an example of one or more embodiments described herein, in which an ingredient listing 1300 is provided for an improved formulation that includes lurasidone as an active ingredient. Such a formulation allows dosage requirements to be stepped down, side effects to be mitigated, as well as overcoming bitterness and/or gustatory issues.

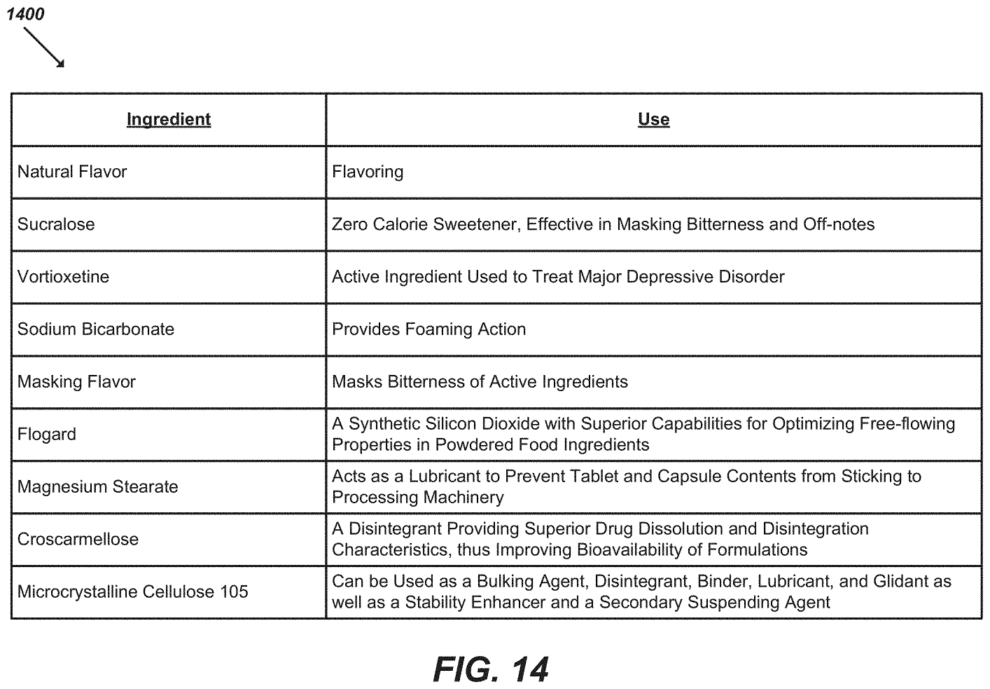

FIG. 14 illustrates an example of one or more embodiments described herein, in which an ingredient listing 1400 is provided for an improved formulation that includes Vortioxetine as an active ingredient. Such a formulation allows dosage requirements to be stepped down, side effects to be mitigated, as well as overcoming bitterness and/or gustatory issues.

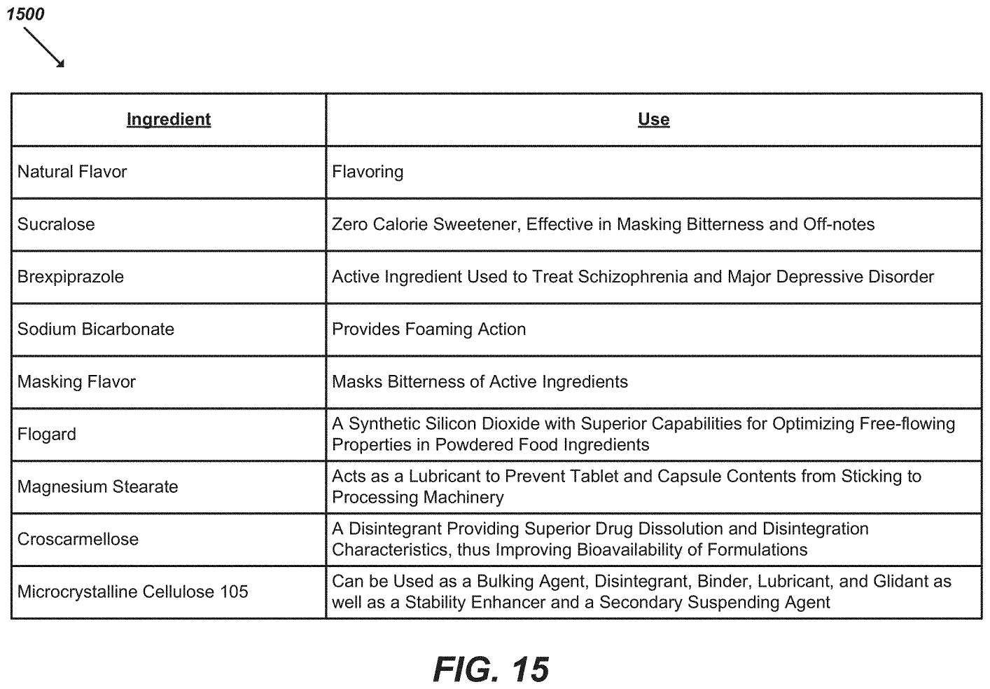

FIG. 15 illustrates an example of one or more embodiments described herein, in which an ingredient listing 1500 is provided for an improved formulation that includes Brexpiprazole as an active ingredient. Such a formulation allows dosage requirements to be stepped down, side effects to be mitigated, as well as overcoming bitterness and/or gustatory issues.

One of ordinary skill in the art will recognize that the ingredient listings 900, 1000, 1100, 1200, 1300, 1400, and/or 1500 are provided as examples and may be varied in different ways without departing from the scope of the disclosure. For instance, similar formulations may be used for various different active ingredients or combinations thereof. As another example, different flavorings and/or sweeteners may be used. As still another example, different lubricants or disintegrants may be used.

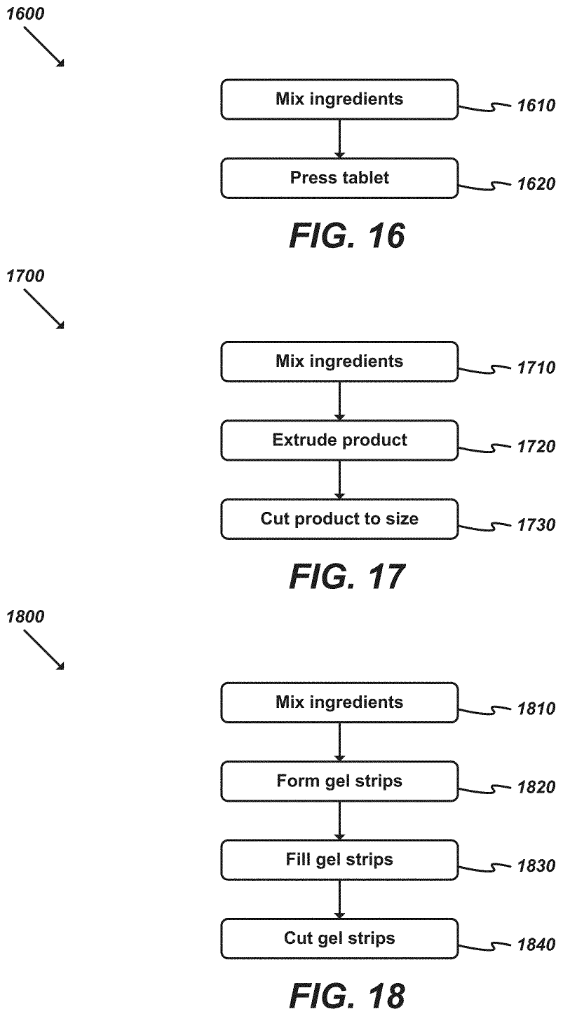

FIG. 16 illustrates an example process 1600 for producing a sublingual tablet. Such a process may be used to make various pressed tablets, such as those described herein.

As shown, process 1600 may include mixing (at 1610) ingredients. Formulations, or sets of ingredients to be mixed, may include any of the substances, compounds, and/or ingredients (whether active or inactive) described herein, and/or other appropriate substances received from various appropriate resources. In some embodiments, the active ingredients may include olanzapine and metformin. In some embodiments, the active ingredients may include blonanserin.

Ingredients may be mixed in various appropriate ways depending on form (e.g., powder, liquid, etc.). In some embodiments, ingredients may be processed before mixing. For example, a liquid substance may be dried to produce a powder. As another example, a powder may be milled to a finer grain. As still another example, a lubricant may be added to a dry powder blend. Ingredients may be mixed to form a homogenous mixture.

Process 1600 may include pressing (at 1620) the sublingual tablet. Tablets may be compressed into a mold or other appropriate resource. Tablets may be sized and shaped in various different ways, depending on relevant factors such as dosage, dissolving time, etc.

Tablets may be pressed using a tablet press or other appropriate devices. Such a tablet press may include a hopper for receiving a power mixture and a cavity formed by a die, a lower punch, and an upper punch. As described above, some embodiments may include convex surfaces while other embodiments may include concave surfaces that may allow saliva to pool. Either type of surface may provide more surface area for absorption than a flat surface.

FIG. 17 illustrates an example process 1700 for extruding a sublingual capsule. Such a process may be used to make various extruded SDVs, such as the gelatin capsules described herein.

As shown, process 1700 may include mixing (at 1710) ingredients. Formulations, or sets of ingredients to be mixed, may include any of the substances, compounds, and/or ingredients (whether active or inactive) described herein, and/or other appropriate substances received from various appropriate resources. In some embodiments, the active ingredients may include olanzapine and metformin. In some embodiments, the active ingredients may include blonanserin. Ingredients may be mixed in various appropriate ways depending on form (e.g., powder, liquid, etc.). In some embodiments, ingredients may be processed before mixing. For example, a liquid substance may be dried to produce a powder. As another example, a powder may be milled to a finer grain. As another example, a powder may be dissolved in liquid. As yet another example, a liquid may be heated.

Process 1700 may include extruding (at 1720) the sublingual product. As discussed above, extruded capsules and/or gel strips may include dry powder and/or liquid or gel fillings that are extruded into a casing such as a gelatin casing. As described above, the extruded sublingual product may include extruded plugs or seals (e.g., gelatin plugs).

The process may include cutting (at 1730) the product to size. Extruded sublingual products may be sized differently based on various relevant factors (e.g., desired dosage, release or absorption time, etc.). The diameter or strip dimensions may be set by an extrusion die, while the length may be varied to achieve different sizes with the same extrusion die.

FIG. 18 illustrates an example process 1800 for manufacturing sublingual gel strips. Such a process may be used to manufacture gel strips including various ingredients, such as those described herein.

As shown, process 1800 may include mixing (at 1810) ingredients. Formulations, or sets of ingredients to be mixed, may include any of the substances, compounds, and/or ingredients (whether active or inactive) described herein, and/or other appropriate substances received from various appropriate resources. In some embodiments, the active ingredients may include olanzapine and metformin. In some embodiments, the active ingredients may include blonanserin.

Process 1800 may include forming (at 1820) gel strips. As described above, gel strips may be formed using an imprint wheel such as imprint wheel 710 to form sheets of cells. Alternatively, gel strips, such as gel strip 840, may be formed by applying pressurized air to a gelatin mixture to generate a gel strip rope, such as rope 810.

The process may include filling (at 1830) the gel strips. Each of the cells may be filled with one or more sets of ingredients including dry powder and/or liquid fillings. After the cells are filled, process may include adding a cover (e.g., a gel cover) to the sheet of cells.

Process 1800 may include cutting (at 1840) the gel strips. The filled sheets may be cut to generate various differently sized strips including a desired number of doses (e.g., a strip of six cells in a single row, a five-by-five set of cells, etc.).

One of ordinary skill in the art will recognize that processes 1000-1800 may be implemented in various different ways without departing from the scope of the disclosure. For instance, the elements may be implemented in a different order than shown. As another example, some embodiments may include additional elements or omit various listed elements. Elements or sets of elements may be performed iteratively and/or based on satisfaction of some performance criteria. Non-dependent elements may be performed in parallel.

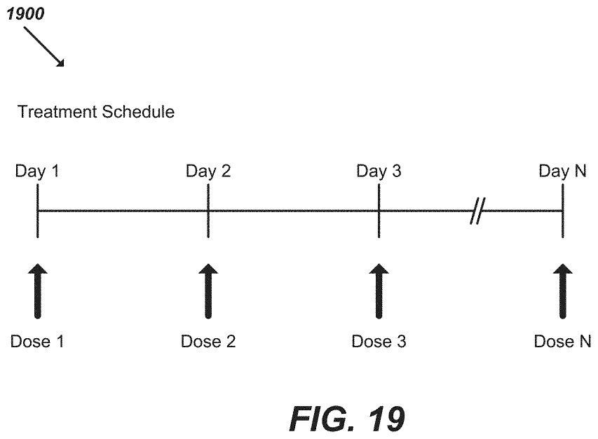

FIG. 19 illustrates an exemplary treatment schedule 1900 that uses sublingual products of some embodiments. Such a treatment schedule 1900 may be associated with, for instance, a sublingual product (e.g., a sublingual capsule or gel strip) including olanzapine and metformin. Such a sublingual product may include, for example, two-and-a-half to fifteen milligrams of olanzapine and five to fifteen milligrams of metformin. As another example, the dosing schedule may be associated with a sublingual product including blonanserin. Such a sublingual product may include, for example, one hundred fifty to four hundred milligrams of blonanserin.

In this example, the treatment schedule includes a single dose administered once per day, for any specified number of days. Different embodiments may be associated with various different treatment schedules, depending on various relevant factors such as dose amount, product type, patient attributes, active ingredient(s), etc. For example, in some embodiments, olanzapine and metformin may be associated with a treatment schedule that includes doses administered twice per day.

No element, act, or instruction used in the present application should be construed as critical or essential unless explicitly described as such. An instance of the use of the term “and,” as used herein, does not necessarily preclude the interpretation that the phrase “and/or” was intended in that instance. Similarly, an instance of the use of the term “or,” as used herein, does not necessarily preclude the interpretation that the phrase “and/or” was intended in that instance. Also, as used herein, the article “a” is intended to include one or more items and may be used interchangeably with the phrase “one or more.” Where only one item is intended, the terms “one,” “single,” “only,” or similar language is used. Further, the phrase “based on” is intended to mean “based, at least in part, on” unless explicitly stated otherwise.

The foregoing relates to illustrative details of exemplary embodiments and modifications may be made without departing from the scope of the disclosure. Even though particular combinations of features are recited in the claims and/or disclosed in the specification, these combinations are not intended to limit the possible implementations of the disclosure. In fact, many of these features may be combined in ways not specifically recited in the claims and/or disclosed in the specification. For instance, although each dependent claim listed below may directly depend on only one other claim, the disclosure of the possible implementations includes each dependent claim in combination with every other claim in the claim set.

We claim:

1. A sublingual delivery vehicle (SDV) comprising:
an ingredient mixture including a flavoring agent and a lubricant; and
a specified dose of lurasidone,
wherein the SDV dissolves within thirty seconds of sublingual administration.

2. The SDV of claim 1, wherein the SDV is a tablet and the ingredient mixture is a dry powder that comprises sodium bicarbonate.

3. The SDV of claim 1, wherein the SDV is a capsule comprising a gelatin casing and the ingredient mixture includes a dry powder, liquid, or gel filling.

4. The SDV of claim 1, wherein the SDV is a gel strip comprising a gelatin cell and gelatin cover and the ingredient mixture includes a dry powder, liquid, or gel filling.

5. The SDV of claim 1, wherein the ingredient mixture comprises a gelatin mixture and the SDV is a bubbled gel strip generated by applying pressurized air to the gelatin mixture.

6. The SDV of claim 1, wherein the specified dose of lurasidone is greater than or equal to ten milligrams and the specified dose of lurasidone is less than or equal to sixty milligrams.

7. The SDV of claim 1, wherein the specified dose of lurasidone is greater than or equal to twenty milligrams and the specified dose of lurasidone is less than or equal to one hundred twenty milligrams.

8. A method of manufacturing a sublingual delivery vehicle (SDV), the method comprising:
mixing a set of ingredients, wherein the set of ingredients comprises:
a flavoring agent,
a lubricant, and
a specified dose of lurasidone; and
forming the SDV from the mixed set of ingredients.

9. The method of claim 8, wherein the ingredient mixture comprises dry powder, wherein the dry powder comprises sodium bicarbonate, and wherein forming the SDV from the mixed set of ingredients includes compressing the ingredient mixture into a tablet.

10. The method of claim 8, wherein the ingredient mixture includes a dry powder, liquid, or gel filling, and wherein forming the SDV from the mixed set of ingredients comprises extruding the ingredient mixture to form a capsule that includes a gelatin casing.

11. The method of claim 8, wherein the ingredient mixture includes a dry powder, liquid, or gel filling, and wherein forming the SDV from the mixed set of ingredients comprises pressing a set of cells into a gelatin sheet, filling each cell from the set of cells with the ingredient mixture, attaching a gel cover to the gelatin sheet, and cutting the gelatin sheet into strips.

12. The method of claim 8, wherein the ingredient mixture comprises a gelatin mixture, and wherein forming the SDV from the mixed set of ingredients comprises applying pressurized air to the gelatin mixture to generate a rope, and cutting the rope into gel strips.

13. The method of claim 8, wherein the specified dose of lurasidone is greater than or equal to ten milligrams and the specified dose of lurasidone is less than or equal to sixty milligrams.

14. The method of claim 8, wherein the specified dose of lurasidone is greater than or equal to twenty milligrams and the specified dose of lurasidone is less than or equal to one hundred twenty milligrams.

15. A method for treating mental disorders, the method comprising:
administering, once a day, an ingredient mixture comprising:
a flavoring agent;
a lubricant; and
a specified dose of lurasidone,
wherein the ingredient mixture is administered via a sublingual delivery vehicle (SDV).

16. The method of claim 15, wherein the SDV is a tablet and the ingredient mixture is a dry powder that comprises sodium bicarbonate.

17. The method of claim 15, wherein the SDV is a capsule comprising a gelatin casing and the ingredient mixture includes a dry powder, liquid, or gel filling.

18. The method of claim 15, wherein the SDV is a gel strip comprising a gelatin cell and gelatin cover and the ingredient mixture includes a dry powder, liquid, or gel filling.

19. The method of claim 15, wherein the ingredient mixture comprises a gelatin mixture and the SDV is a bubbled gel strip generated by applying pressurized air to the gelatin mixture.

20. The method of claim 15, wherein the specified dose of lurasidone is greater than or equal to ten milligrams and the specified dose of lurasidone is less than or equal to sixty milligrams.
